(19) United States
(12) Patent Application Publication
(10) Pub. No.: US 2011/0282428 A1
(43) Pub. Date: Nov. 17, 2011

(54) BIODEGRADABLE COMPOSITE STENT

(75) Inventors: MICHAEL P. MEYER, Richfield, MN (US); DOUG THESINGH, Minneapolis, MN (US)

(73) Assignee: BOSTON SCIENTIFIC SCIMED, INC., Maple Grove, MN (US)

(21) Appl. No.: 13/093,240

(22) Filed: Apr. 25, 2011

Related U.S. Application Data
(60) Provisional application No. 61/334,329, filed on May 13, 2010.

(51) Int. Cl. A61F 2/82

(52) U.S. Cl. 623/1.15

(57) ABSTRACT

A biodegradable stent body is defined by a composite of a biodegradable polymer and a biodegradable metal insert. The composition, geometry, and location of the metal and polymer are selected for desirable stent performance.

### CROSS-REFERENCE TO RELATED APPLICATION

This application claims priority under 35 USC §119(e) to U.S. Provisional Patent Application Ser. No. 61/334,329, filed on May 13, 2010, the entire contents of which are hereby incorporated by reference.

### TECHNICAL FIELD

The present description relates to endoprostheses, and more particularly to stents.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

It is sometimes desirable for an implanted endoprosthesis to erode over time within the passageway. For example, a fully erodable endoprosthesis does not remain as a permanent object in the body, which may help the passageway recover to its natural condition. Erodible endoprostheses can be formed from, e.g., a polymeric material, such as polylactic acid, or from a metallic material such as magnesium, iron or an alloy thereof

### SUMMARY

The present description is directed to an endoprosthesis, such as, for example, a biodegradable stent.

In an aspect, the description features a stent including a stent body composed of a biodegradable polymer case and a biodegradable metal insert within the case, the body including a high bending strength region having a bending strength contributed predominantly by the insert and lower bending strength region having a tensile strength contributed primarily by the polymer.

In an aspect, the description features a stent body defined by a series of undulating struts that are composed of a biodegradable polymer case and a biodegradable metal insert, wherein the thickness of the insert is between about 30-80% of the stent thickness and the relative thickness of the case and the insert thickness varies in different regions of the stent body.

Embodiments may include one or more of the following features. The bending strength of the insert is 80% or more of the bending strength of the high bending strength region. The bending strength of the case is 80% or more of the bending strength of the lower bending strength region. The bending strength of the case is 100% of the bending strength of the lower bending strength region. The insert is thicker in the high bending strength region than in the low bending strength region. The insert has a maximum thickness of about 50-75% of the thickness of the high bending strength region. The case is thicker in the low bending strength region than the high bending strength region.

The case has a maximum thickness of about 80-100% of the thickness of the low bending strength region. The stent body includes a series of undulating struts including substantially linear segments joined by corner segments, and the corner segments are high bending regions and the linear segments are lower bending regions. The inserts are thicker at the corner segments than in the linear segments. The linear segments include inserts. The inserts are thicker at apices of the corner segments. The linear segments do not include inserts. The inserts are plastically deformed upon expansion of the stent when the case is plastically deformed upon expansion of the stent. The metal inserts are formed of Mg, Fe, Zn, Mn, stainless steel, nitinol, cobalt, chromium, platinum, or an alloy including two or more of Mg, Fe, Zn Mn, chromium, platinum, cobalt, nitinol, and stainless steel. The polymer case is formed of PLA, PLGA, POSS, or Tyrosine-Polcarbonate.

Embodiments may include one or more of the following features. The insert is non-continuous within the case. The insert is completely encapsulated by the case. The insert is partially encapsulated by the case.

Embodiments may include one or more of the following advantages. Different case and insert materials are combined to achieve optimum mechanical performance for bioerodible stents. Polymers can have the benefit of being more biocompatible and erode in a more controlled fashion (e.g. they can be engineered to erode at a given rate which might change over time) and the ability to more controllably contain and elute active agents, but can lack significant strength and rigidity in the necessary dimensions for a vascular stent. By combining a metal (or metal-like) core with a polymer exterior, the polymer is reinforced and the metal component is kept small to enhance surface area to mass ratio so that the component is readily erodible and also to minimize the effects of erosion, such as local tissue irritation and high levels of degredant. In embodiments, the stent body includes a biodegradable polymer case and a biodegradable metal insert. The relative dimensions, geometry, composition and location of these components are selected in coordination to provide desirable performance characteristics. In particular embodiments, the inserts are provided, and made stronger and thicker in or adjacent to the areas of highest stress, such as the apices of undulating stent features or adjacent the apices. For example, the insert can be thicker closer to an apex and thinner or even absent in less stressed, e.g., linear strut regions. The resulting stent can maintain lumen patency for a desired period, while providing a desired bioerosion profile. The stent can be also used to strengthen a weakened vessel, e.g. an aneurysm, treat intrastent restenosis in a previously implanted stent, treat chronic vasospasm, or vulnerable unstable plaque and stimulate natural healing. A nonpermanent stent can also reduce the need for follow on surgery or other therapy and reduce interference with imaging techniques such as MRI. The stent can substantially reduce recoil after expansion compared to a stent not including inserts.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C are sequential, longitudinal cross-sectional views, illustrating delivery of an endoprosthesis in a collapsed state, expansion of the endoprosthesis, and the deployment of the endoprosthesis in a body lumen.

FIG. 2 is a perspective view of an embodiment of a stent.

FIG. 3A is an expanded view of region of a stent body.

FIG. 3B is a perspective view of an apex region of a strut in a compressed condition.

FIG. 3C is a perspective view of the apex region of strut in an expanded state.

FIG. 3D is an assembly view of an apex region of a strut.

FIG. 4A is a perspective view of a stent body illustrating stress concentration, while FIG. 4B is an expanded view of a strut region in FIG. 4A.

FIG. 5 is a view of a region of a stent body.

FIGS. 6A-6G illustrate insert arrangements.

FIGS. 7A and 7B are views of a region of a stent body.

FIG. 8A illustrates a stent region, while FIG. 8B is a cross-section along lines AA.

FIGS. 9A and 9B illustrate other insert arrangements.

### DETAILED DESCRIPTION

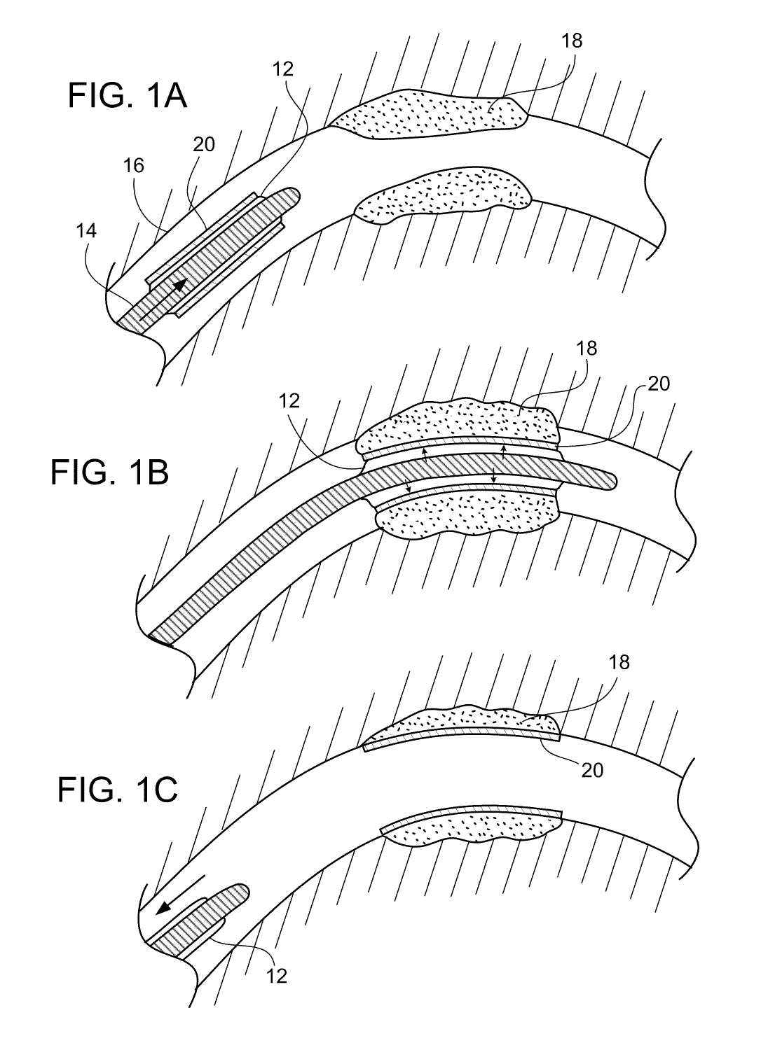

Referring to FIGS. 1A-1C, a stent 20 is placed over a balloon 12 carried near a distal end of a catheter 14, and is directed through the lumen 15 (FIG. 1A) until the portion carrying the balloon and stent reaches the region of an occlusion 18. The stent 20 is then radially expanded, e.g. by inflating the balloon 12, and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C). In other embodiments, the stent can be an elastic or superelastic. Stents are further described in Heath, U.S. Pat. No. 6,290,721.

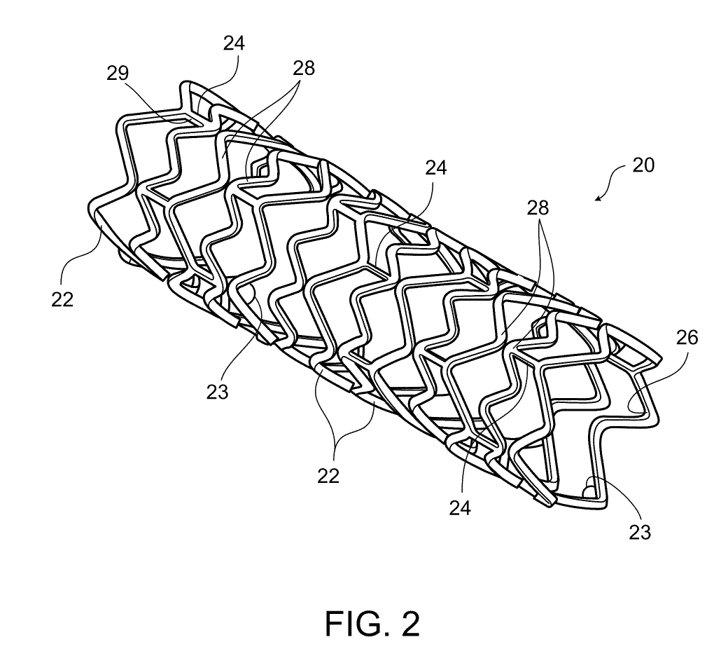

Referring to FIG. 2, an expandable stent 20 can have a stent body having the form of a tubular member defined by a plurality of struts that includes undulating bands 22 and a plurality of connectors 24 that extend between and connect adjacent bands. During use, bands 22 can be expanded from an initial, smaller diameter to a larger diameter to contact stent 20 against a wall of a vessel, thereby maintaining the patency of the vessel. Connectors 24 can provide stent 20 with flexibility and conformability that allow the stent to adapt to the contours of the vessel. One or more bands 22 form acute angles 23 at corner regions. The angle 23 increases upon expansion of the stent. Stent body 20, bands 22 and connectors 24 can have a luminal surface 26, an abluminal surface 28, and a sidewall surface 29. In embodiments, the bands and/or connectors, have a width, W, and a thickness, T, of about 50 to 150 microns. The struts can include non-linear designs between apices. Connectors can provide uniformity of coverage and repeatable length; they also affect flexibility, conformability, and fatigue resistance. The W and T may be identical or different, may vary along the length of the member and/or along the axial length of the entire device. The W and T may form a repeating pattern and may or may not be symmetric. Suitable stent designs include the Liberte available from Boston Scientific, Natick, Mass.

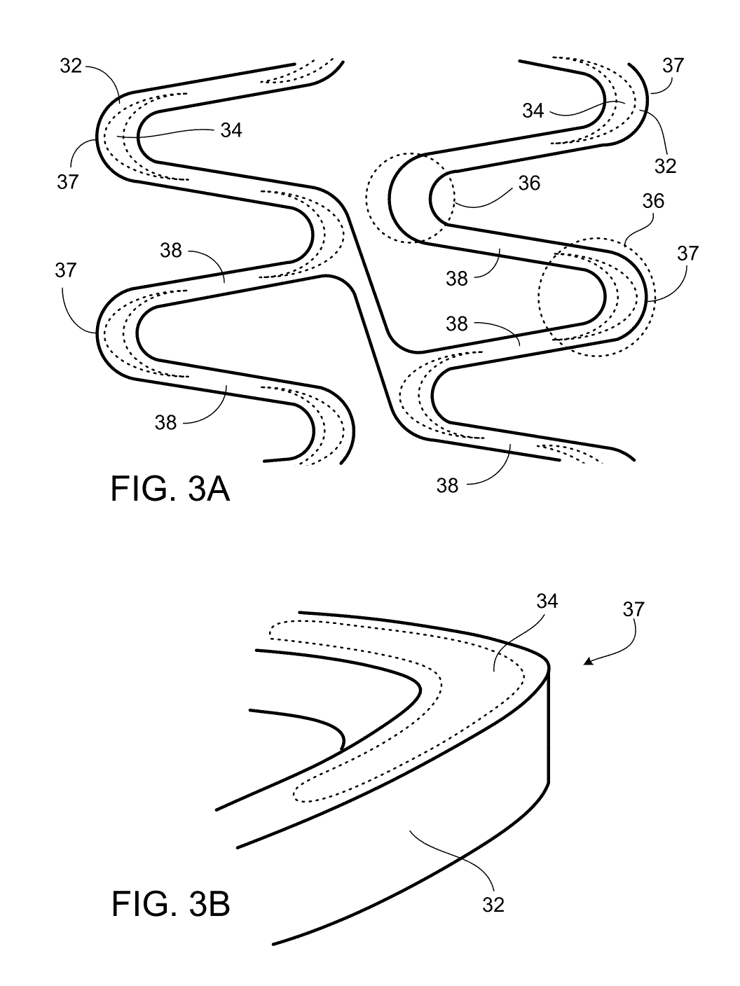

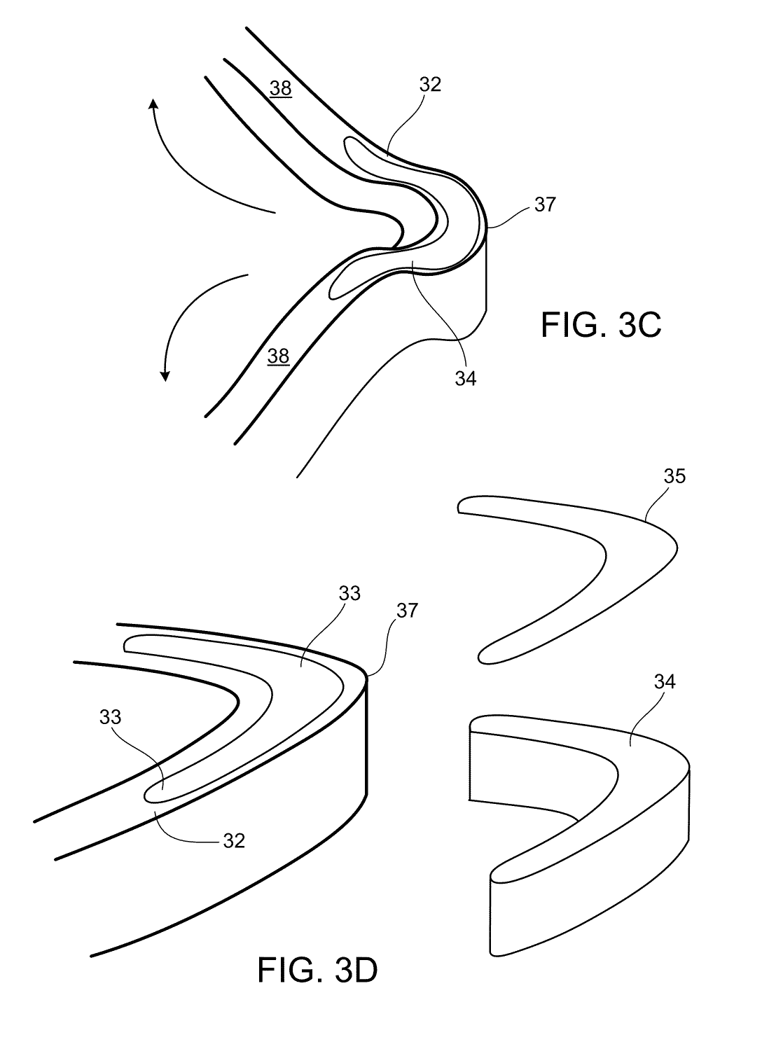

Referring to FIG. 3A, the struts are composed of a composite of a biodegradable outer polymer case 32 and within the case 32 biodegradable metal inserts 34 (as shown in phantom). The struts can include a series of corner regions 36 with apices 37 which join adjacent regions 38 which can be substantially linear or have other configurations. Referring as well to FIG. 3B, the metal inserts 34 are provided in the corner regions, which as illustrated in FIG. 3C, undergo substantial stress and strain as they are deformed for delivery and during expansion of the stent upon deployment.

The composition, location, and geometry of the metal inserts 34 and the polymer cases 32 are determined in coordination to provide desirable properties such as the mechanical strength required to maintain patency of the lumen and desirable bioerosion characteristics, such that the stent biodegrades in a desirable period after deployment, and reducing recoil of the stent after expansion. In the embodiment illustrated in FIGS. 3A-3D, the inserts are discrete, non-continuous elements located in regions of highest stress such as in and adjacent to the corner regions. The inserts have a nonuniform thickness profile. In this embodiment, the inserts are thicker near the peak of the apices 37, and thinner spaced from the apices 37. In this particular embodiment, the inserts 34 are crescent shaped. In portions of the linear regions 38, the inserts 34 are absent and the strength of the stent body is provided entirely by the polymer case 32. In other embodiments, the insert has other geometries that span the corner shape and/or inserts are provided in the regions 38. The non-continuous metal inserts with polymer sections in between provides enhanced MRI compatibility with less visual artifact, heating, displacement, and torque effects. In the embodiment of FIGS. 3A-3D, the inserts are completely encapsulated by the polymer case such that the insert is not exposed to the body upon implantation. In other embodiments, the inserts are partially encapsulated by the case, e.g. on two sides, allowing the metal to be exposed to the body on the other two sides.

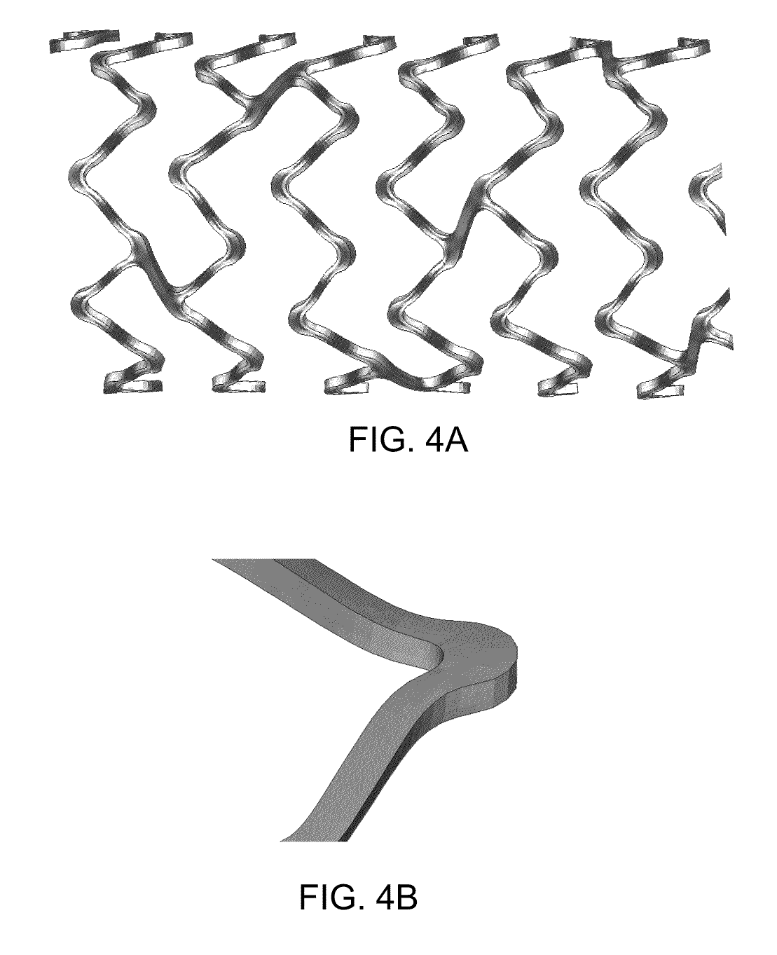

Referring to FIGS. 4A and 4B, the stress regions of an expanded stent are illustrated. Darker shades indicate lower stress and lighter shades indicate higher stress. In embodiments, insert material is provided in high stress regions having two or more times the stress lowest stress regions, e.g. 3-5× the lowest stress regions. In embodiments, the lowest stress regions are free of inserts. In other embodiments, inserts may span the lower stress regions to higher stress regions. Stress analysis can be conducted by finite element analysis using software available from Ansys, Canonsburg, Pa.

In embodiments, when the stent is expanded, the metal insert is plastically deformed and cold-worked, allowing for sufficient rigidity and strength. This allows the stent to behave more like an all-metal stent initially upon implant. Later, after vascular remodeling, less stent strength is needed. At this time point, the polymer layer may have delivered a drug and eroded, leaving a fully-exposed and weakened metal insert. This insert, due to its small dimensions and high surface-area-to-mass ratio, would then be susceptible to rapid bio-erosion or bio-absorption while achieving minimal vessel irritation due to the limited material present for corrosion. Premature fracture of the case, prior to the ISO-mandated 380M pulsate cycles, is less of a concern such that the dimensions of the insert member may be optimized for acute performance and bio-absorption rather than long-term mechanical integrity. In embodiments, the use of inserts substantially reduces recoil of a stent. When a stent is expanded and the expansion force removed, stents can sometimes “recoil” to a smaller diameter. With the use of inserts as described here, recoil can be reduced by 5% or more, e.g. 10 to 15% or more or 25% or less, compared to a similar polymer stent without inserts.

The tensile and/or yield and/or bending strength of the composite can be increased in high stress regions, such as regions about the apices. In particular embodiments, the bending strength is increased in high stress regions. A higher bending strength is a measure of both higher yield strength and a higher force needed per unit of deflection. In embodiments, the bending strength in high stress regions is contributed predominately, e.g. 80% or more, by the inserts, whereas in lower stress regions the bending strength may be contributed predominantly, or entirely, by the polymer case.

In embodiments, the cross sectional thickness of the inserts in the high stress regions is about 30-80% of the thickness of the strut and the cross sectional thickness of the case in the lower stress regions is up to 100% of the thickness of the strut. In addition, in embodiments, the dimensions of the insert create regions of little or no bending (apices), regions of bending and plastic deformation of the material (transition), and regions of bending and elastic deformation of the material (central portion of strut).

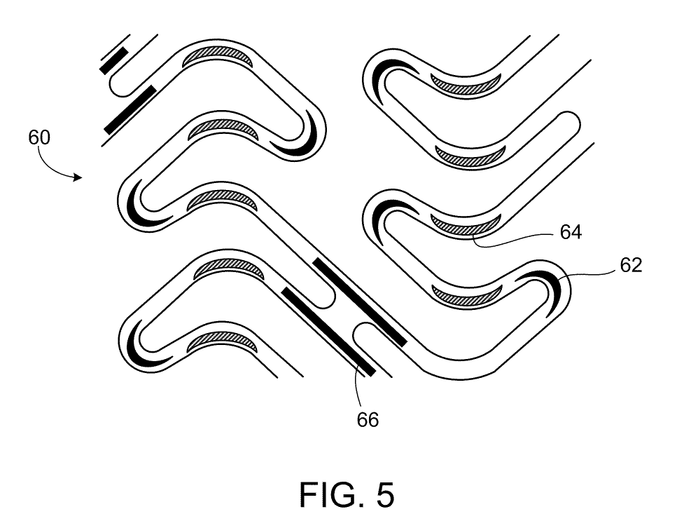

Referring to FIG. 5, inserts of different types can be used in the same stent. A stent 60, for example, can include a case 61 and inserts 62, 64, 66 at different locations and/or of different geometries and material, depending on amount of strain or degradation rate desired in specific areas of the stent. For example, insert 62 is provided about a small radial apex and is formed of a high bending strength material in the shape of a crescent. Insert 64 is provided around a larger radius, lower stress apex and is formed of a lower bending strength metal in the shape of a shallower crescent. Inserts 66 are provided in substantially linear sections, about connectors and are formed of high tensile metal in a generally linear geometry. In embodiments, the inserts 66 are designed, e.g., by selection of materials, shapes, dimensions, locations, and others, to deform upon deployment of the stent 60 to reduce recoiling and provide strength to the stent 60. In embodiments, inserts can be made of radiopaque materials and could be used as markers on struts to aid visibility. The radiopaque markers can be only on end segments for visibility and are left behind to mark treatment area after stent has eroded.

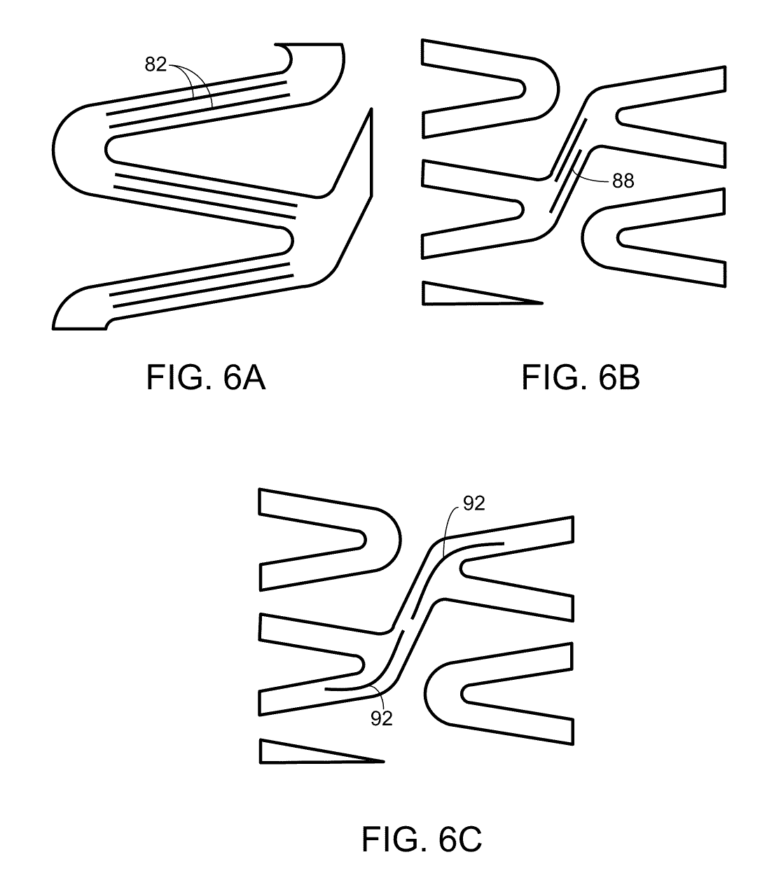

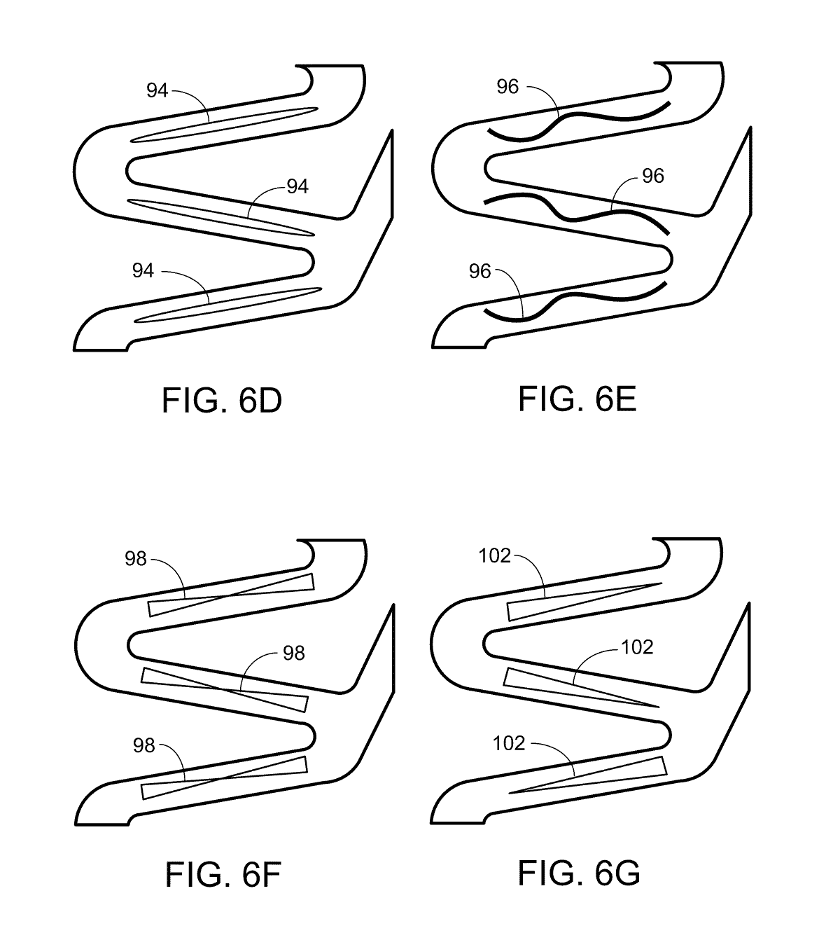

Referring to FIGS. 6A-6G, other insert geometries are shown. Referring to FIG. 6A, inserts 82 are provided as substantially linear, parallel elements radially offset from the axis of a linear portion of a strut. Referring to FIG. 6B, the inserts 88 are parallel and offset. Referring to FIG. 6C, inserts 92 extend along a bonded portion of a strut. The inserts 86 and/or 92 can provide strength to the connectors of the stents (e.g., connector 24 of FIG. 2). Referring to FIG. 6D, inserts 94 taper from the center of a linear region of the strut. The inserts 94 can provide additional strength to the center of the struts. Referring to FIG. 6E, inserts 96 have a sinusoidal shape such that the distance from the surface of the stent body varies. The sinusoidal shape, or other possible shapes not shown, can be selected and used in selected regions of the stents, e.g., based on the local strain in the particular regions of the stents. For example, the inserts can vary its positions and shapes throughout the stent. Referring to FIG. 6F, inserts 98 have an hour glass shape. Referring to FIG. 6G, inserts 102 have a triangular shape. The various shapes, e.g., width of the inserts, e.g., inserts 98 and inserts 102, can be selected to accommodate the different local strains in various locations of the stents. One or more features of the inserts or one or more inserts described in FIGS. 6A-6G can be selected and combined for use in one stent to provide the stent with desired combined features and advantages.

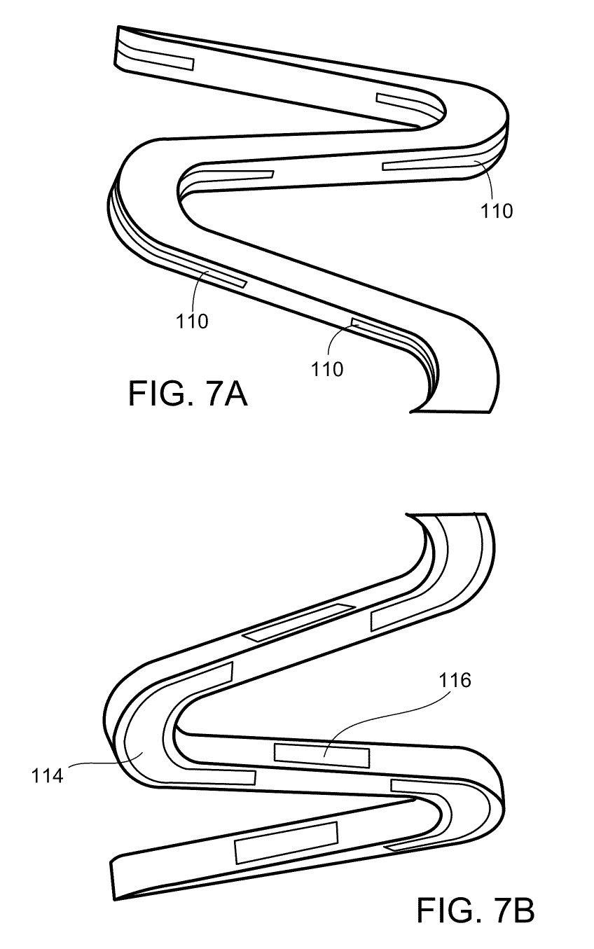

Referring to FIG. 7A, a stent is illustrated in which inserts 110 are exposed on the side wall surfaces of the strut. Referring to FIG. 7B, a stent is illustrated in which different inserts are exposed on different surfaces. In particular, inserts 114 about the apices are exposed as luminal and abluminal surfaces. Inserts 116 in linear regions are exposed on the side walls.

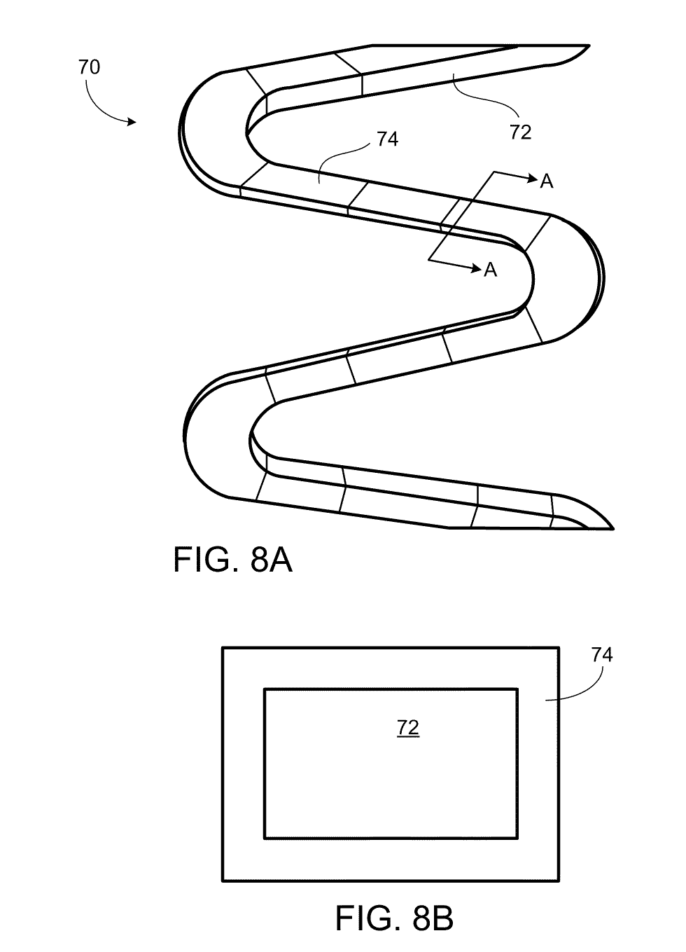

Referring to FIGS. 8A and 8B, in other embodiments, a stent 70 includes a polymer body 72 and inserts 74 in the form of metal bands intermittently wrapped around the body 72. The metal can also be vapor deposited onto the body 72 and can be positioned at the apices.

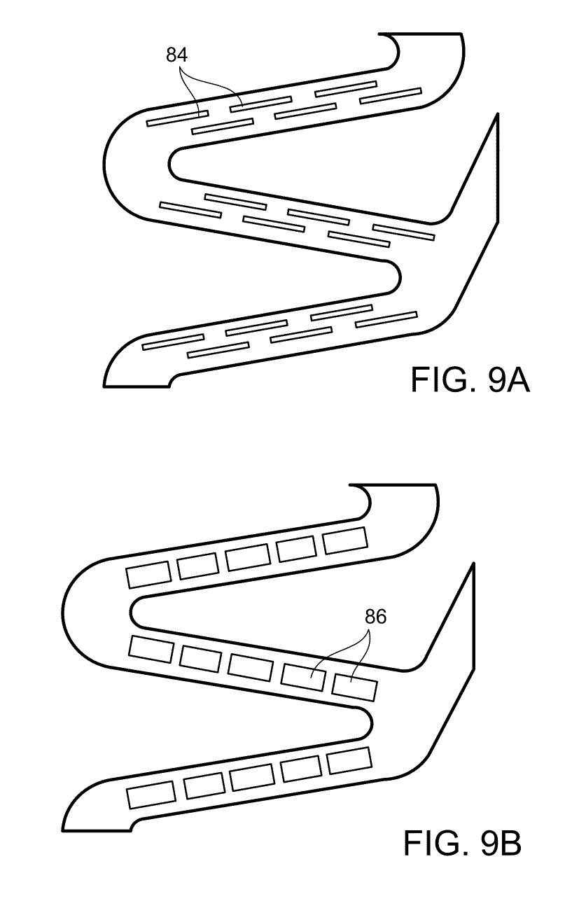

Referring to FIG. 9A, a series of inserts 84 are arranged similar to FIG. 6A but are provided as a series of discrete segments. Referring to FIG. 9B, inserts 86 are provided as a series of discrete segments along the axis. The sizes, shapes, and numbers of the discrete segments can be varied, e.g., based on design needs. In some embodiments, inserts in the form of the discrete segments (as shown in FIGS. 9A and 9B) provide less strength more flexibility to the stent portion than those including continuous the inserts (e.g., inserts of FIGS. 6A). For example, a stent may include expandable rings connected by the connectors (e.g., FIG. 2) and the inserts in the form of discrete segments can be used in the connectors to provide both strength and flexibility to the stent, e.g., to facilitate the stent to conform to the tortuous vessels in a body. Different inserts, e.g., those shown in FIGS. 6A-6G and 9A-9B can be selected based on the need of a particular stent and can be used in different parts of a single stent.

Suitable metals for inserts include Mg and Fe, alloys including these elements, and other soluble metallic ions present in the body such as Zn or Mn. Suitable polymers include PLA, PLGA or polyhedral oligosilsesquioxane (POSS) or tyrosine-based polycarb or others (available from REVA, Inc.). In embodiments, the metal is non-degradable. Suitable metals also include stainless steel, nitinol, cobalt, chromium, platinum, and the alloy thereof, e.g., the alloy of chromium and platinum. Other suitable materials are described below. The insert can be manufactured using techniques such as laser cutting from pre-fabricated tubing, stamping, molding or other machining The surrounding polymer case can then be formed using insert molding, with or without a subsequent material removal step (e.g. laser cutting), spray application, roll application, dipping, etc. Multiple applications could be done for multiple layers. Referring back to FIG. 3D, for example, the polymer case 38 can be preformed and void areas 33 formed, e.g. by laser application, into which the insert 34 can be friction fit and/or fitted with an adhesive and polymer cover 35 is then positioned over the insert and bonded to the polymer case 38.

### OTHER EMBODIMENTS

A stent is bioerodible if the stent or a portion thereof exhibits substantial mass or density reduction or chemical transformation, after it is introduced into a patient, e.g., a human patient. Mass reduction can occur by, e.g., dissolution of the material that forms the stent and/or fragmenting of the stent. Chemical transformation can include oxidation/reduction, hydrolysis, substitution, and/or addition reactions, or other chemical reactions of the material from which the stent or a portion thereof is made. The erosion can be the result of a chemical and/or biological interaction of the stent with the body environment, e.g., the body itself or body fluids, into which it is implanted. The erosion can also be triggered by applying a triggering influence, such as a chemical reactant or energy to the stent, e.g., to increase a reaction rate. For example, a stent or a portion thereof can be formed from an active metal, e.g., Mg or Fe or an alloy thereof, and which can erode by reaction with water, producing the corresponding metal oxide and hydrogen gas; a stent or a portion thereof can also be formed from a bioerodible polymer, or a blend of bioerodible polymers which can erode by hydrolysis with water. Fragmentation of a stent occurs as, e.g., some regions of the stent erode more rapidly than other regions. The faster eroding regions become weakened by more quickly eroding through the body of the endoprosthesis and fragment from the slower eroding regions.

Preferably, the erosion occurs to a desirable extent in a time frame that can provide a therapeutic benefit. For example, the stent may exhibit substantial mass reduction after a period of time when a function of the stent, such as support of the lumen wall or drug delivery, is no longer needed or desirable. In certain applications, stents exhibit a mass reduction of about 10 percent or more, e.g. about 50 percent or more, after a period of implantation of about one day or more, about 60 days or more, about 180 days or more, about 600 days or more, or about 1000 days or less. Erosion rates can be adjusted to allow a stent to erode in a desired sequence by either reducing or increasing erosion rates. For example, regions can be treated to increase erosion rates by enhancing their chemical reactivity, e.g., coating portions of the stent with a silver or zinc coating to create a galvanic couple with the exposed, uncoated Iron surfaces on other parts of the stent.. Alternatively, regions can be treated to reduce erosion rates, e.g., by using coatings. Suitable materials are also discussed in Jabera et al., “Bioabsorbable Stents: The Future is Near”, Cardiac Interventions Today, p. 50, June/July 2009.

A coating can be deposited or applied over the surface of stent to provide a desired function. Examples of such coatings include a tie layer, a biocompatible outer coating, a radiopaque metal or alloy, and/or a drug-eluting layer. A stent can be incorporated with at least one releasable therapeutic agent, drug, or pharmaceutically active compound to inhibit restenosis, such as paclitaxel, or to treat and/or inhibit pain, encrustation of the stent or sclerosing or necrosing of a treated lumen. The therapeutic agent can be a genetic therapeutic agent, a non-genetic therapeutic agent, or cells. The therapeutic agent can also be nonionic, or anionic and/or cationic in nature. Examples of suitable therapeutic agents, drugs, or pharmaceutically active compounds include anti-thrombogenic agents, antioxidants, anti-inflammatory agents, anesthetic agents, anti-coagulants, and antibiotics, as described in U.S. Pat. No. 5,674,242; U.S. Ser. No. 09/895,415, filed Jul. 2, 2001 and published as U.S. 2003/0003220; U.S. Ser. No. 11/111,509, filed Apr. 21, 2005 and published as U.S. 2005/0251249; and U.S. Pat. No. 7,462,366, the entire disclosure of each of which is herein incorporated by reference. Representative conventional approaches disperse the therapeutic agent, drug, or a pharmaceutically active compound in a polymeric coating carried by a stent. In the present invention, the therapeutic agent, drug, or a pharmaceutically active compound can be directly incorporated into the pores generated by plasma immersion ion implantation treatment on the surface of a stent, thereby eliminating the use of extra coatings.

The stent body or as a layer on a stent made of another material, or can include a layer of another material, which other material may be bioerodible or biostable, a metal, a polymer or a ceramic. The stent can include in addition to the materials described above. In some embodiments, the stent can include one or more bioerodible metals, such as magnesium, zinc, iron, or alloys thereof. The stent can include bioerodible and non-bioerodible materials. The stent can have a surface including bioerodible metals, polymeric materials, or ceramics. The stent can have a surface including an oxide of a bioerodible metal. Examples of bioerodible alloys also include magnesium alloys having, by weight, 50-98% magnesium, 0-40% lithium, 0-1% iron and less than 5% other metals or rare earths; or 79-97% magnesium, 2-5% aluminum, 0-12% lithium and 1-4% rare earths (such as cerium, lanthanum, neodymium and/or praseodymium); or 85-91% magnesium, 6-12% lithium, 2% aluminum and 1% rare earths; or 86-97% magnesium, 0-8% lithium, 2-4% aluminum and 1-2% rare earths; or 8.5-9.5% aluminum, 0.15%-0.4% manganese, 0.45-0.9% zinc and the remainder magnesium; or 4.5-5.3% aluminum, 0.28%-0.5% manganese and the remainder magnesium; or 55-65% magnesium, 30-40% lithium and 0-5% other metals and/or rare earths. Bioerodible magnesium alloys are also available under the names AZ91D, AM50A, and AE42. Other bioerodible alloys are described in Bolz, U.S. Pat. No. 6,287,332 (e.g., zinc-titanium alloy and sodium-magnesium alloys); Heublein, U.S. Patent Application 2002000406; and Park, Science and Technology of Advanced Materials, 2, 73-78 (2001), the entire disclosure of each of which is herein incorporated by reference. In particular, Park describes Mg—X—Ca alloys, e.g., Mg—Al—Si—Ca, Mg—Zn—Ca alloys. Examples of bioerodible polymers include polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-lactide, poly-D-lactide, polyglycolide, poly(alpha-hydroxy acid), and combinations thereof.

A stent can also include non-bioerodible materials. Examples of suitable non-bioerodible materials include stainless steels, platinum enhanced stainless steels, cobalt-chromium alloys, nickel-titanium alloys, noble metals and combinations thereof. In some embodiments, stent 20 can include bioerodible and non-bioerodible portions. In some embodiments, non-bioerodible or biostable metals can be used to enhance the X-ray visibility of bioerodible stents. The bioerodible stent main structure of a stent can be combined with one or more biostable marker sections. The biostable marker sections can include, for example, Gold, Platinum or other high atomic weight elements. The biostable marker sections can provide enhance visibility and radiopacity and can provide a structural purpose as well.

A stent can have any desired shape and size (e.g., superficial femoral artery stents, coronary stents, aortic stents, peripheral vascular stents, gastrointestinal stents, urology stents, and neurology stents). Depending on the application, stent 20 can have an expanded diameter of about 1 mm to about 46 mm. For example, a coronary stent can have an expanded diameter of about 2 mm to about 6 mm; a peripheral stent can have an expanded diameter of about 5 mm to about 24 mm; a gastrointestinal and/or urology stent can have an expanded diameter of about 6 mm to about 30 mm; a neurology stent can have an expanded diameter of about 1 mm to about 12 mm; and an abdominal aortic aneurysm stent and a thoracic aortic aneurysm stent can have an expanded diameter of about 20 mm to about 46 mm. Stent 20 can be self-expandable, balloon-expandable, or a combination of self-expandable and balloon-expandable (e.g., as described in U.S. Pat. No. 5,366,504). Stent 20 can have any suitable transverse cross-section, including circular and non-circular (e.g., polygonal such as square, hexagonal or octagonal).

A stent can be implemented using a catheter delivery system. Catheter systems are described in, for example, Wang U.S. Pat. No. 5,195,969; Hamlin U.S. Pat. No. 5,270,086; and Raeder-Devens, U.S. Pat. No. 6,726,712, the entire disclosure of each of which is herein incorporated by reference. Commercial examples of stents and stent delivery systems include Radius®, Symbiot® or Sentinol® system, available from Boston Scientific Scimed, Maple Grove, Minn.

A stent can be a part of a covered stent or a stent-graft. For example, a stent can include and/or be attached to a biocompatible, non-porous or semi-porous polymer matrix made of polytetrafluoroethylene (PTFE), expanded PTFE, polyethylene, urethane, or polypropylene. In addition to vascular lumens, a stent can be configured for non-vascular lumens. For example, it can be configured for use in the esophagus or the prostate. Other lumens include biliary lumens, hepatic lumens, pancreatic lumens, uretheral lumens and uretheral lumens. MOF's as discussed above, e.g. as a layer for drug delivery, can be utilized on other implantable medical devices such as pacing and defibrillation leads.

The insert could be manufactured using current techniques (e.g. laser cutting from pre-fabricated tubing). The surrounding polymer layer(s) could then be added using insert molding, with or without a subsequent material removal step (e.g. laser cutting), spray application, roll application, dipping, etc. Multiple applications could be done for multiple layers. Polymer portion of the stent can be impregnated with drug or the stent could be coated after processing by common drug coating methods such as spray coating, roll-coating, or LabCoat methods. In another embodiment the main body of the stent and the metal peaks could have different drugs or doses. The metal inserts could be press fit into place or secured by alternative bonding mechanisms such as degradable adhesives.

All references, such as patent applications, publications, and patents, referred to herein are incorporated by reference in their entirety.

Still further embodiments are in the following claims.

What is claimed is:

1. A stent, comprising:
a stent body composed of a biodegradable polymer case and a biodegradable metal insert, the body including a high bending strength region having a bending strength contributed predominantly by the insert and a lower bending strength region having a bending strength contributed primarily by the polymer.

2. The stent of claim 1 wherein the bending strength of the insert is 80% or more of the bending strength of the high bending strength region.

3. The stent of claim 1 wherein the bending strength of the case is 80% or more of the bending strength of the lower bending strength region.

4. The stent of claim 3 wherein the bending strength of the case is 100% of the bending strength of the lower bending strength region.

5. The stent of claim 1 wherein the insert is thicker in the high bending strength region than in the low bending strength region.

6. The stent of claim 5 wherein the insert has a maximum thickness of about 50-75% of the thickness of the high bending strength region.

7. The stent of claim 1 wherein the case is thicker in the low bending strength region than the high bending strength region.

8. The stent of claim 5 wherein the case has a maximum thickness of about 80-100% of the thickness of the low bending strength region.

9. The stent of claim 1 wherein the stent body includes a series of undulating struts including substantially linear segments joined by corner segments, and wherein the corner segments are high bending regions and the linear segments are lower bending regions.

10. The stent of claim 9 wherein the inserts are thicker at the corner segments than in the linear segments.

11. The stent of claim 9 wherein the linear segments include inserts.

12. The stent of claim 11 wherein the inserts are thicker at apices of the corner segments.

13. The stent of claim 9 wherein the linear segments do not include inserts.

14. The stent of claim 1 wherein the inserts are plastically deformed upon expansion of the stent when the case is plastically deformed upon expansion of the stent.

15. The stent of claim 1 wherein the metal inserts are formed of Mg, Fe, Zn Mn, stainless steel, nitinol, cobalt, chromium, platinum, or an alloy including two or more of Mg, Fe, Zn Mn, chromium, platinum, cobalt, nitinol, and stainless steel.

16. The stent of claim 1 wherein the polymer case is formed of PLA, PLGA, POSS, or Tyrosine-Polcarbonate.

17. A stent body composed of a series of undulating struts that are composed of a biodegradable polymer case and a biodegradable metal insert, wherein the thickness of the insert being between about 50-75% of the stent thickness and the relative thickness of the case and the insert thickness varies in different regions of the stent body.

18. The stent of claim 17 wherein the insert is non-continuous within the case.

19. The stent of claim 17 wherein the insert is completely encapsulated by the case.

20. The stent of claim 17 wherein the insert is partially encapsulated by the case.
